(19) United States
(12) Patent Application Publication
(10) Pub. No.: US 2025/0174328 A1
(43) Pub. Date: May 29, 2025

(54) SYSTEM FOR AUTOMATICALLY PRESCRIBING AND/OR ADJUSTING INSULIN DOSES FOR A PATIENT

(71) Applicants: UNIVERSITY OF PITTSBURGH-OF THE COMMONWEALTH SYSTEM OF HIGHER EDUCATION, PITTSBURGH, PA (US); UPMC, PITTSBURGH, PA (US)

(72) Inventors: DAVID ROMETO, PITTSBURGH, PA (US); SANDRA INDACOCHEA SOBEL, PITTSBURGH, PA (US)

(73) Assignees: UNIVERSITY OF PITTSBURGH-OF THE COMMONWEALTH SYSTEM OF HIGHER EDUCATION, PITTSBURGH, PA (US); UPMC, PITTSBURGH, PA (US)

(21) Appl. No.: 18/840,143

(22) PCT Filed: Feb. 28, 2023
(86) PCT No.: PCT/US2023/063399

§ 371 Date: Aug. 21, 2024

Related U.S. Application Data
(60) Provisional application No. 63/268,711, filed on Mar. 1, 2022.

(51) Int. Cl. G16H 20/10; G16H 40/67

(52) U.S. Cl. CPC G16H 20/10; G16H 40/67

(57) ABSTRACT

A system for prescribing insulin doses is provided, wherein a first titration application component structured to is implemented on a patient computing device of the patient, wherein the first titration application component logs glucose level data measured from the patient and insulin dose data indicative of a number of insulin doses given to the patient. The system includes a provider computing including a second titration application component, wherein the second titration application component is structured and configured to receive the glucose level data and the insulin dose data from the patient computing device, determine a correction factor based on a sensitivity constant specified by the healthcare provider, and determine the insulin dose recommendation for the patient based on the insulin dose data, the glucose level data and the correction factor, wherein the provider computing device is structured and configured to transmit the insulin dose recommendation to the patient computing device.

### FIELD OF THE INVENTION

The present invention pertains to the management of diabetes, and, in particular, to a system and associated method(s) for enabling the automatic prescription and/or adjustment of insulin doses for a patient by a healthcare provider of the patient.

### BACKGROUND OF THE INVENTION

Currently in the practice of diabetes care, medical providers verbally and in writing provide patients with insulin doses at or between appointments. This may also include a “correction scale”, listing several glucose ranges followed by a number of units of insulin to take when the glucose falls into that range. American Diabetes Association guidelines do recommend having a patient increase their scheduled dose by a small amount 1-2 times/week until a specific glucose target is met for basal insulin (for instance, have a patient start on 10 units, and if they increase 2 units once a week, they may get to the needed 50 units to achieve fasting glucose under 130 mg/dL in 20 weeks). But in practice, this is not routinely asked of patients, and when it is, the patient often fails to follow through until target glucose is reached.

After starting on insulin, patients typically continue to have glucose levels above goal for months to years, and providers may only review glucose log data or A1C blood test results every 3 to 6 months to make a safe upward adjustment. In high-risk pregnancy and some endocrinology clinics, providers will review glucose logs weekly and make adjustments to get glucose levels to goal very quickly, requiring a lot of staff time and patient compliance with paper logging and returning logs to clinics. In those with type 1 or 2 diabetes staying on insulin long term, the doses that achieve control change over time and fluctuate up and down over months and years.

Currently available technology does not include insulin dose titration features. Insulin pumps that integrate with continuous glucose monitors (mostly in use by patients with type 1 diabetes) have bolus calculators that provide insulin dose based on current glucose and meal carb content, then adjust basal rate continuously based on glucose level and trend, some giving small correction boluses hourly. The prescribed settings themselves do not automatically change, nor does the device suggest that they change when glucose goals are consistently not met.

### SUMMARY OF THE INVENTION

In one embodiment, a system for automatically prescribing and/or adjusting insulin doses for a patient is provided, wherein a first titration application component structured to is implemented on a patient computing device of the patient, wherein the first titration application component is structured and configured to log glucose level data measured from the patient and to log insulin dose data indicative of a number of insulin doses given to the patient. The system includes a provider computing device of a healthcare provider of the patient, the provider computing device including a second titration application component structured to be implemented on the provider computing device, wherein the second titration application component is structured and configured to receive the glucose level data and the insulin dose data from the patient computing device, determine a correction factor based on a sensitivity constant specified by the healthcare provider, and determine the insulin dose recommendation for the patient based on the insulin dose data, the glucose level data and the correction factor, wherein the provider computing device is structured and configured to transmit the insulin dose recommendation to the patient computing device.

In another embodiment, a system for automatically prescribing and/or adjusting a basal insulin dose for a patient is provided, wherein a first titration application component is implemented on a patient computing device of the patient, wherein the first titration application component is structured and configured to log: (i) existing basal dose data indicative of an existing basal dose for the patient, (ii) glucose change data for the patient indicative of a change between a bedtime glucose level of the patient and a breakfast glucose level of the patient, and (iii) time of drop data indicative of a time period over which the change occurred. The system in this embodiment includes a provider computing device of a healthcare provider of the patient, the provider computing device including a second titration application component structured to be implemented on the provider computing device, wherein the second titration application component is structured and configured to receive the existing basal dose data, the glucose change data, and the time of drop data, determine an insulin dose recommendation for the patient based on existing basal dose data, the glucose change data and the time of drop data, and transmit the insulin dose recommendation to the patient computing device.

### BRIEF DESCRIPTION OF THE DRAWINGS

FIG. 1 is a schematic diagram of a system for automatically prescribing and/or adjusting insulin doses according to an exemplary embodiment of the disclosed concept;

FIG. 2 is a schematic diagram of a patient computing device forming a part of the system of FIG. 1 according to an exemplary embodiment of the disclosed concept; and

FIG. 3 is a schematic diagram of provider computing device forming a part of the system of FIG. 1 according to an exemplary embodiment of the disclosed concept.

### DETAILED DESCRIPTION

As used herein, the singular form of “a”, “an”, and “the” include plural references unless the context clearly dictates otherwise.

As used herein, the statement that two or more parts or components are “coupled” shall mean that the parts are joined or operate together either directly or indirectly, i.e., through one or more intermediate parts or components, so long as a link occurs.

As used herein, the term “number” shall mean one or an integer greater than one (i.e., a plurality).

As used herein, the terms “component” and “system” are intended to refer to a computer related entity, either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution, and a component can be localized on one computer and/or distributed between two or more computers. While certain ways of displaying information to users are shown and described with respect to certain figures or graphs as screenshots, those skilled in the relevant art will recognize that various other alternatives can be employed.

As used herein, the term “controller” shall mean a programmable analog and/or digital device (including an associated memory part or portion) that can store, retrieve, execute and process data (e.g., software routines and/or information used by such routines), including, without limitation, a field programmable gate array (FPGA), a complex programmable logic device (CPLD), a programmable system on a chip (PSOC), an application specific integrated circuit (ASIC), a microprocessor, a microcontroller, a programmable logic controller, or any other suitable processing device or apparatus. The memory portion can be any one or more of a variety of types of internal and/or external storage media such as, without limitation, RAM, ROM, EPROM(s), EEPROM(s), FLASH, and the like that provide a storage register, i.e., a non-transitory machine readable medium, for data and program code storage such as in the fashion of an internal storage area of a computer, and can be volatile memory or nonvolatile memory.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

The disclosed concept will now be described, for purposes of explanation, in connection with numerous specific details in order to provide a thorough understanding of the disclosed concept. It will be evident, however, that the disclosed concept can be practiced without these specific details without departing from the spirit and scope of this innovation.

As described in greater detail herein, in the exemplary embodiment, the disclosed concept provides a mobile (e.g., phone) application that allows a medical provider to prescribe a complex insulin dose adjustment algorithm to their patient. The patient then uses the application from a provided patient-interface to log glucose results, receive dose recommendations, and log doses given. The disclosed concept can be used in various types of diabetes, and in patients using once daily basal insulin only or basal plus rapid pre-meal insulin, and/or in patients checking glucose once daily, 4 times daily, 7 times daily, or wearing a continuous glucose monitor. The disclosed concept also provides a provider-interface that includes a settings section that allows the provider to choose several parameters including initial doses, target high and low glucoses before and after meals, the rate of dose increases and decreases, the magnitude of and maximum and minimum dose increases and decreases for meal doses and basal insulin, and an insulin sensitivity factor and target for correction doses.

The application of the disclosed concept, with doses provided to the patient that can go up daily until specific targets are reached and can be viewed by the provider at any time without patient initiation, solves many of the problems of existing diabetes solutions described herein. In the exemplary embodiment, as long as the patient has a cellular signal and a method of checking their current glucose level, they can be under the watchful care of a provider who can adjust targets and intensity of the algorithm titration at any time. The patient thus does not need to make adjustments to the dose or doses directly.

Other features of the disclosed concept include adjusting pre-meal and correction doses with continuous glucose monitor trend data, presented in “arrows” up or down. A down trending arrow indicating that the glucose level is already on its way down will result in a lower insulin dose given for that meal, to prevent hypoglycemia. Also, if settings are for the patient to check glucose on his or her finger or a CGM 2 hours after meals, an alert with be sent to the patient 2 hours after they confirmed their pre-meal dose to remind them to check and enter post-meal glucose.

In the exemplary embodiment, the application of the disclosed concept works by using glucose level data, insulin dose data, and CGM arrow data, if available, from the current day and the past 1-7 days, to provide the patient user with the dose of insulin to give before the current meal or the long acting insulin daily. The methodology used to create the suggested dose uses the patient's total daily dose (TDD) from the day before, a sensitivity constant specified in the provider settings (and used to determine a sensitivity factor as described herein), the current glucose level, and the current trend arrow(s) for correction doses with arrow adjustments. The current meal-specific pre-meal base dose is determined by the change in glucose from before said meal to 2 or 4+ hours later, for the day or days prior. The linear equation y=mx+b is used to adjust the dose given the day before (that included the glucose and arrow correction) up or down by a percentage. The slope and intercept of the equations, as well as the maximum and minimum increase and decrease percentages, determine how aggressively the doses can be changed, and therefore how long it will take to achieve the adequate doses. These are set by the prescribing provider in a clinically clear format.

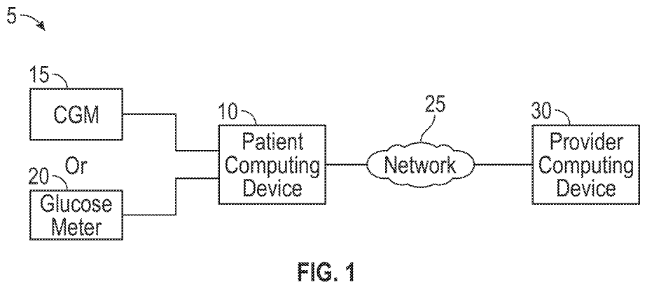

FIG. 1 is a schematic diagram of a system 5 for automatically prescribing and/or adjusting insulin doses for a diabetes patient according to an exemplary embodiment of the disclosed concept. Referring to FIG. 1, system 5 includes a patient computing device 10, such as, without limitation, a smartphone, a tablet computer, a laptop computer or a PC, that belongs to the diabetes patient. System 5 also includes one or more tools for measuring the current glucose level of the patient. For example, such tools may include a continuous glucose monitor (CGM) 15 that is worn on the patient's body and that is structured and configured to measure the blood glucose levels of the patient and transmit that data (in a wired or wireless fashion) to patient computing device 10. Alternatively, such tools may include a glucose meter 20 by which the patient is able to measure blood glucose levels from a small blood sample provided by the patient, typically by way of a finger prick. The blood glucose levels measured by glucose meter 20 are able to be provided (in a wired or wireless fashion) to patient computing device 10 (or by manual simple input). System 5 further includes a communications network 25, such as, without limitation, the Internet. Finally, system 5 includes a provider computing device 30 that may be accessed by a healthcare provider of the patient. As seen in FIG. 1, two-way data communication between patient computing device 10 and provider computing device 30 as described in further detail herein is enabled through communications network 25 in a wired and/or wireless fashion.

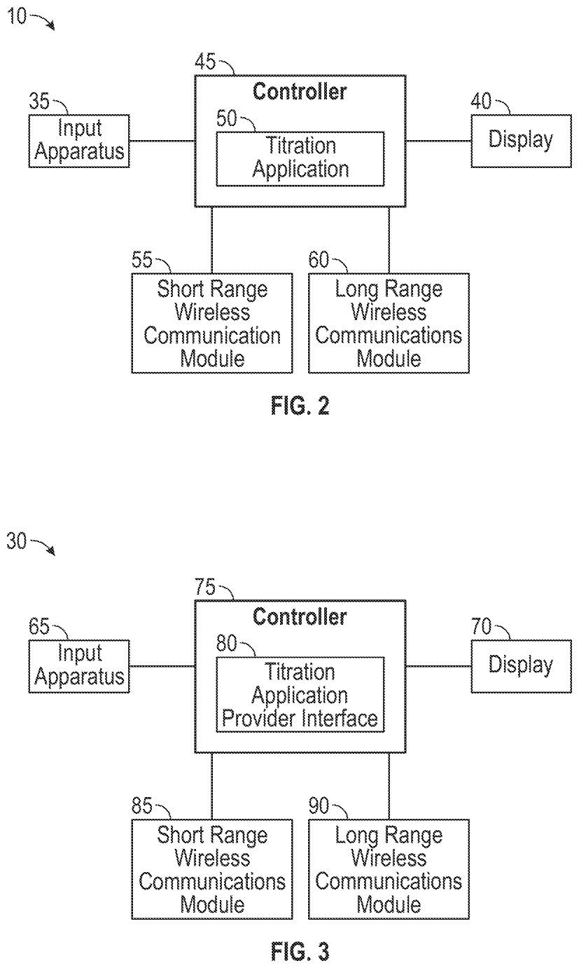

FIG. 2 is a schematic diagram of patient computing device 10 according to an exemplary embodiment of the disclosed concept as described herein. The exemplary patient computing device 10 is, for illustrative purposes, a smartphone, although it will be appreciated that patient computing device 10 may be another other type of electronic device, such as, without limitation, a tablet computer, a laptop computer or a PC, without departing from the scope of the disclosed concept. As seen in FIG. 2, patient computing device 10 includes an input apparatus 35, which may include a plurality of buttons and/or a touchscreen, a display 40, and a controller 45. A user (e.g., the patient) is able to provide input into controller 45 using input apparatus 35, and controller 45 provides output signals to display 40 to enable display 40 to display information to the patient as described herein.

As described elsewhere herein, controller 45 includes a processor portion and a memory portion. The memory portion has stored therein a number of routines that are executable by the processor portion. According to an aspect of the disclosed concept, controller 45 includes one or more routines that implement (by way of computer/processor executable instructions) a titration application 50 that is configured to, among other things, enable the collection of data relating to the diabetes care of the patient and the prescription and/or adjustment of insulin doses for the patient by a healthcare provider according to one or more of the various embodiments described in detail herein. In the exemplary embodiment, titration application 50 may be downloaded to patient computing device 10 from any suitable source, such as an online “app store.”

As seen in FIG. 2, patient computing device 10 also includes a short range wireless communications module 55 that is structured and configured to enable patient computing device 10 to communicate with other, similarly equipped electronic devices over a short range wireless network. In the exemplary embodiment, short range wireless communications module 55 is a Bluetooth® module that that is structured and configured to enable patient computing device 10 to communicate with other devices over an ad hoc Bluetooth® network, and subsequently over network 25. Patient computing device 10 also includes a long range wireless communications module 60 that is structured and configured to enable patient computing device 10 to communicate with other electronic devices and systems over network 25. As will be appreciated, the communication modules just described enable patient computing device 10 to be in electronic communication with provider computing device 30 in order to perform the various functions that are described in detail herein.

FIG. 3 is a schematic diagram of provider computing device 30 according to an exemplary embodiment of the disclosed concept. The exemplary provider computing device 30 is, for illustrative purposes, a PC, although it will be appreciated that provider computing device 30 may be another other type of electronic device, such as, without limitation, a tablet computer, a laptop computer or a smartphone, without departing from the scope of the disclosed concept. As seen in FIG. 3, provider computing device 30 includes an input apparatus 65, which may include a plurality of buttons and/or a touchscreen, a display 70, and a controller 75. A healthcare provider of the patient is able to provide input into controller 75 using input apparatus 65, and controller 75 provides output signals to display 70 to enable display 70 to display information to the healthcare provider as described in detail herein.

Controller 75 includes a processor portion and a memory portion. The memory portion has stored therein a number of routines that are executable by the processor portion. According to an aspect of the disclosed concept, controller 75 includes one or more routines that implement (by way of computer/processor executable instructions) a titration application provider interface 80 that is configured to, among other things, enable the healthcare provider to receive and review data relating to the diabetes care of the patient and specify and provide instructions, which are communicated to the patient computing device 10, for the prescription and/or adjustment of insulin doses for the patient.

As seen in FIG. 3, provider computing device 30 also includes a short range wireless communications module 85 and a long range wireless communications module 90 that are similar in function to the short went range wireless communications module 55 and long range wireless communications module 60, respectively, described elsewhere herein. As will be appreciated, such communication modules enable provider computing device 30 to be in electronic communication with patient computing device 10 in order to perform the various functions that are described in detail herein.

Referring again to FIG. 1, in operation, patient computing device 10 receives glucose level data from the patient from either CGM 15 or glucose meter 24 for the current day and for a previous period, such as, without limitation, the past 1-7 days. In addition, in the case where CGM 15 is used, patient computing device 10 will also receive CGM arrow data indicating the current trend in the patient's insulin levels. Patient computing device 10 then transmits such data, along with the current insulin dose data for the patient, to provider computing device 30 via network 25. Upon receipt of that data at provider computing device 30, the healthcare provider is able to view that data and therefore make dosing decisions based thereon. In particular, healthcare provider will employ a novel and innovative titration algorithm as described in greater detail herein in order to determine the recommended dose for the patient based on the received data and certain other data that is input into titration application provider interface of 80 of controller 75. In the exemplary embodiment, such algorithm is implemented as part of titration application provider interface 80 such that the determination of the recommended dose is performed in an automated manner. In particular, and as described in greater detail in various exemplary embodiments herein, the algorithm determines the suggested dose for the patient using several parameters including, without limitation, the patient's total daily dose from the day before, a sensitivity constant provided in the provider settings (and used to determine sensitivity factor as described herein), the patient's current glucose, and, if available, CGM trend arrow data. As noted elsewhere herein, in the exemplary embodiment, a linear equation is used to adjust the dose given the day before up or down by a percentage. The slope and intercept of the equation, as well as the maximum and minimum increase and decrease percentages, determine how aggressively the doses can be changed, and therefore how long it will take to achieve the adequate dose. Such parameters may be specified by the healthcare provider using titration application provider interface 80. Once the recommended dose is determined, data indicative thereof is transmitted by provider computing device 30 to patient computing device 10 via network 25. In response, the patient is able to make the recommended change and/or adjustment to their insulin dosing based upon the received information.

According to a further aspect of the disclosed concept, the algorithm employed by titration application provider interface 80 includes a methodology for determining basal insulin levels based on certain parameters discussed herein, and a separate methodology for determining meal correction/arrow adjustment insulin doses based on certain parameters discussed herein. Thus, system 5 enables the patient's healthcare provider to prescribe and/or adjust insulin doses for the patient in the form of both basal insulin doses and meal correction/and arrow adjustment insulin doses as needed without the need for the patient to make any determinations and/or do any calculations on their own.

### Basal Insulin Methodology

As noted above, one aspect of the disclosed concept relates to a methodology for adjusting basal insulin doses based on certain parameters received from patient computing device 10 and/or input into provider computing device 30. Provided below is a detailed description of one particular implementation of such a methodology according to one non-limiting exemplary embodiment of the disclosed concept. According to this methodology, a recommended basal dose of a long-acting insulin, such as glargine, is provided for dosing in the AM for daily titration of basal bolus to tight targets. The methodology described may be implemented in titration application 50 shown in FIG. 2 and/or in titration application provider interface 80 shown in FIG. 3.

The described methodology relies upon several parameters that are specified by the healthcare provider, which, in the exemplary embodiment, is done by way of titration application provider interface 80. Specifically, the exemplary titration application provider interface 80 includes: (i) a “Settings” page for specifying the following inputs: starting dose, high fasting glucose target, low fasting glucose target, whether the patient is on ultra-long acting basal, frequency of dose increases (in days), whether the patient provides insulin in whole unit pen/syringe doses, half-unit pen/syringe doses, or by an insulin pump, whether the patient follows a consistent carb diet or instead practices carb counting using a carb ratio, and whether basal insulin is given before breakfast or after dinner; (ii) an “Advanced Settings” page for specifying the following inputs: max basal increase %, min basal increase %, min basal decrease %, and fasting glucose over target at max, and (iii) a “Dosing” page for specifying the following inputs: the patient's fasting glucose and basal dose given. The exemplary titration application provider interface 80 also includes a “Dosing” page output that provides the recommended basal dose (determined as described below) as an output.

The following describes the basal dose calculation according to this aspect of the disclosed concept in the case where such dosing is to start the day after the starting dose is administered, if the frequency of dose increase is set at 1 day. In particular, the dose calculation is determined as described below based on three different possible patient conditions (as indicated by information from the patient computing device 10. Those three conditions are (i) fasting glucose above high target, (ii) fasting glucose between high and low target, and (iii) fasting glucose below low target.

For fasting glucose above high target, the new recommended dose is calculated as a percent increase from the previous day's dose calculated by multiplying the previous day's dose by a factor Y (that is >1.0), from the linear equation Y=mx+b, where X is the magnitude of glucose above the high target (fasting glucose-high target, always positive), referred to herein as “fasting glucose over target at max”, b is the minimum increase factor for any glucose above the high target (e.g., 1.1 for a 10% increase), referred to herein as “basal increase y-intercept”, and m is the slope of the line, referred to herein as “basal increase slope”. Moreover, the basal increase slope m is calculated from the equation Y=mX+b, using Y=1+(basal max increase %)/100, X=fasting glucose over target at max, and basal increase y-intercept b=1+(basal min increase %)/100. Therefore, m=(Y−b)/X, or m={[1+(basal max increase %)/100]−[1+(basal min increase %)/100]}/(fasting glucose over target at max). Based on the above, the recommended dose=the previous day's dose×[basal increase slope×(fasting glucose over target at max)+basal increase y-intercept]. In the exemplary embodiment, there is a set max percent increase for the recommended dose, so for example, if this is set at 15%, then even if the calculation gives a result of Y=1.2, the recommended dose would still be to increase as if Y=1.15.

The following is an example of this determination for an exemplary patient using actual numbers. Suppose a patient currently taking 45 units of basal wakes up with a glucose of 170, when the fasting glucose target range is set to min=80 (low fasting glucose target), max=130 (high fasting glucose target). So his/her glucose is 170−130, or 40 mg/dL over the max target (fasting glucose over target at max). If the patient's settings are that his/her max basal increase %=50, and his/her min basal increase %=10, and his/her glucose above target at max=70, then his/her current dose of basal insulin would be increased by multiplying 45 units by: 0.00571429×40 mg/dL+1.1=1.32857, where 0.00571429 is the calculated basal increase slope from the formula above. As a result, the recommended does would be 60 units basal insulin that day.

For fasting glucose between high and low target, there will be no change to the recommended dose.

For fasting glucose below low target, the new recommended dose is calculated as a percent decrease from the previous day's dose calculated by multiplying the previous day's dose by a factor Y (that is between 0 and 1.0), from the linear equation Y=mx+b, where X is the magnitude of glucose below the low target (glucose-low target, always negative)), referred to herein as “fasting glucose under target at min”, b is the minimum decrease factor for any glucose below the low target (0.9 for a 10% decrease), referred to herein as “basal decrease y-intercept”, and m is the slope of the line, referred to herein as “basal decrease slope”. There is NO set max decrease, as this would put the patient at risk for recurrent severe hypoglycemia. In this case, in the exemplary embodiment, the basal decrease y-intercept and the basal decrease slope are set by values used during a pilot study conducted by the present inventors, where a 30% reduction in dose was made when fasting glucose was 25 mg/dL below the low target, with a minimum basal decrease factor (Y-intercept b) of 0.9, giving m=0.008. Based on the above, the recommended dose=the previous day's×[basal decrease slope×(fasting glucose under target at min)+basal decrease y-intercept].

Furthermore, in the exemplary embodiment, the present methodology includes a hypoglycemia override. In particular, if patient has a low glucose (below the input value on the settings page) between bedtime and fasting AM entry, and the fasting glucose level is higher than the glucose level during hypoglycemia, the reduction in basal dose will be calculated using the hypoglycemia override formula as follows: Y=0.01333X+0.9, with a 10% decrease for a glucose just below hypoglycemia value, and slope derived from a 30% decrease for a glucose 15 mg/dL below the hypoglycemia value.

Furthermore, according to a further aspect of this methodology, the following describes how the basal dose calculation would be performed for settings of dose increase frequency of 2 days or greater. Specifically, in this circumstance dose decreases are unchanged. Dose increases, however, are calculated by the average of the Y values over that period of days, with a glucose at target generating a Y value of 1.0. As values below target will result in a dose decrease that day, the day-clock starts over at that time. For example, if set to increase every 4 days, and since the last dose change, the patient's fasting glucose was at goal once, and above high target 3 days, with Y values of 1.11, 1.15, and 1.13, then his/her basal dose would be calculated based on the formula from his/her settings page, with a Y of (1.0+1.1+1.15+1.13)/4=1.095. If using ultra long acting basal insulin, 1 and 2 day increase frequency will not be done. 3 days is the lowest frequency for these insulins, and only the averages of the Y values starting on day 3 will be used for adjustments. Decreases for under target will still be made on the first day.

### Mealtime Correction Dose/Arrow Adjustment Dose Methodology

As noted above, another aspect of the disclosed concept relates to a methodology for determining either or both of a mealtime correction does and/or an arrow adjustment dose based on certain parameters received from patient computing device 10 and/or input into provider computing device 30. Both the mealtime correction dose and the arrow adjustment dose, if calculated, would be added to the base dose for a meal that the patient is about to consume. Provided below is a detailed description of one particular implementation of such a methodology according to one non-limiting exemplary embodiment of the disclosed concept.

The described methodology relies upon several parameters that are specified by the healthcare provider, which, in the exemplary embodiment, is done by way of titration application provider interface 80. Specifically, in the current embodiment, the exemplary titration application provider interface 80 allows the following parameters to be specified on the indicated pages (which have been previously described elsewhere herein): (i) the “Settings” page is used for specifying the following inputs: whether the patient is using a CGM (e.g., CGM 15), whether the patient is taking a premeal bolus insulin, whether the patient is checking 2 hr post meal (postprandial or pp) glucose, the initial recommended premeal bolus dose for the patient, a sensitivity constant (e.g., 1500 for a more aggressive correction or 1800 for a less aggressive correction) (as described herein, the provided sensitivity constant is used to determine a sensitivity factor according to various embodiments), a 2 hr pp glucose rise high target, a 2 hr pp glucose rise low target, a next premeal or bedtime high target, a next premeal or bedtime low target, a correction target, and a frequency of meal dose increase; (ii) the “Advanced Settings” page is used for specifying the following inputs: a Meal Max increase %, a Meal Min increase %, a Meal min decrease %, and a Post Meal Glucose over target at max, and (iii) the “Dosing” page is used for specifying the following inputs: the patient's premeal glucose level (received from patient computing device 10 in the exemplary embodiment), the premeal arrow (from a CGM, if used, which indicates how high the glucose will be in 30 minutes if nothing is done), if a meal has been eaten, the dose given, and a 2 hr pp glucose level (measured exactly 2 hours after start of meal). The exemplary titration application provider interface 80 also includes a “Dosing” page output that provides the recommended mealtime correction dose and the arrow adjustment dose (determined as described below), if present, as an output.

In the exemplary embodiment, the mealtime correction dose (which is the increase in mealtime insulin dose due to current hyperglycemia, often called sliding scale or correction insulin) is calculated as follows: mealtime correction dose=(premeal glucose level-correction target)/(a sensitivity factor). As noted above, the sensitivity factor can be determined from the provided sensitivity constant in a number of ways. In one exemplary embodiment, the sensitivity factor (in mg/dL/unit) is calculated as the specified sensitivity constant/total daily dose of insulin (TDD). Using the examples provided herein, the sensitivity factor would be 1500/TDD for a more aggressive correction, and 1800/TDD for a less aggressive correction. In other embodiments, the sensitivity factor (in mg/dL/unit) may calculated as: the specified sensitivity constant/basal dose (i.e., long acting insulin dose), or as: the specified sensitivity constant×a carb ratio that the patient uses for determining proper insulin levels based on carb counting. The “Settings” page may also allow the user to select the manner in which the sensitivity factor is determined. Thus, the “Settings” page inputs include a number, the sensitivity constant, which will be manipulated as described herein to provide the sensitivity factor (in mg/dL/unit), allowing for correction doses to get larger as patient's TDD goes up during titration to the adequate dose for control. Note, in the exemplary embodiment, this adjustment is only made if glucose is ABOVE the correction target; the exemplary embodiment does not include lowering the meal dose for a glucose BELOW the correction target.

Accordingly, in short, in the exemplary embodiment, the mealtime correction dose=(premeal glucose level-correction target)/(sensitivity factor). For example, where sensitivity factor is determined as sensitivity constant/TDD, for a patient with a premeal glucose level of 250 and a correction target of 150, a sensitivity constant of 1500, and a current TDD of 75 units, the mealtime correction dose would be (250−150)/(1500/75)=5 units.

In the exemplary embodiment, the arrow adjustment dose (if the patient is wearing a CGM, such as CGM 15) is calculated in the following manner (this is skipped if the patient is not wearing a CGM). The arrow adjustment dose (which may be positive or negative) is determined by the magnitude and direction of the premeal arrow, which indicates how high the glucose will be in 30 minutes if nothing is done. Each added arrow indicates a proportional increase or decrease based on the patient's sensitivity to insulin, which in the exemplary embodiment is determined by the sensitivity factor as determined from the specified/selected sensitivity constant in any manner described herein. In the exemplary embodiment, a value of 30/sensitivity factor is used for each arrow increment, from −3 to +3. Accordingly, in the exemplary embodiment where the sensitivity factor is determined as sensitivity constant/TDD, the arrow adjustment dose=the number of arrow increments×(30/(sensitivity constant/TDD)). For example, a patient with 2 up arrows (3 increments up), a sensitivity constant of 1800, and a current TDD of 58 units, the arrow adjustment dose would be 3(30/(1800/58))=2.9 units.

As noted elsewhere herein, according to this methodology of the disclosed concept, both the mealtime correction dose and the arrow adjustment dose would be added to the base dose for a given meal. As also noted herein, how the patient doses (e.g., whole units from a pen or syringe, half units from a pen or syringe, or 0.1 units from an insulin pump) can be specified. In the exemplary embodiment, rounding to a whole or half unit should be done after adding the 3 non-rounded values together (and not before). For example, using whole units, a calculated new base dose of 3.3+meal correction dose of 1.3+an arrow adjustment dose of 2.3 would be 6.9, rounded to 7 units, NOT 3+1+2=6, nor 4+2+3=9.

As noted elsewhere herein, the “Settings” page may allow a user to specify that the patient practices carb counting using a carb ratio. In such an embodiment, the insulin dose recommendation for the patient may also be based on the carb ratio instead of base meal dose alone, by using a ratio as follows: a meal carb estimation for a meal provided by the patient/the carb ratio. More specifically, according to this embodiment, adjustment for the next day is made by assuming the carbs in meal divided by the entire bolus given (carb+correction+arrow adjustment) is the carb ratio used in that meal, and that number will be adjusted by the percentage change formula for next day's carb ratio. And percentage change will be opposite direction than for fixed base dose calculations.

As also noted elsewhere herein, the “Settings” page may allow a user to specify whether basal is given before breakfast or after dinner. In such an embodiment, if basal is given before breakfast in a day, then the meal insulin dose recommendation for the patient for that day will be adjusted up or down by ⅙^{th} of the unit adjustment to the basal dose. If basal is given after dinner in a day, then the insulin dose recommendation for the patient for the next day will be adjusted up or down by ⅙^{th} of the unit adjustment to the basal dose.

### Titration of Meal Base Dose for the Next Day

A further aspect of the disclosed concept includes a methodology for titrating the base dose for a meal on a given day based on information from the previous day for those patients that are checking glucose levels 2 hours after meals (on finger or CGM). In particular, the new base dose for a meal on a given day is, in this embodiment, determined by the rise or fall of glucose levels at 2 hours after the same meal (say, breakfast) the day before, in relation to the target increase after 2 hours. But, the dose is determined by adjusting the TOTAL dose they received before that meal (not just the base dose). Thus, if for the same meal the previous day the patient received 5 units as a meal base dose, 2 units as a meal correction dose (as determined herein), and 1 unit for an arrow adjustment dose (8 units in total), and the glucose rise at 2 hours after the meal during that previous day was at goal (e.g., between high and low target), then the meal base dose for the current day for the same meal would be 8 units.

More specifically, according this aspect of the disclose concept, the base dose for a meal on a given day is calculated as described below based on three different possible conditions from the previous day as described herein. Those three conditions are 2 hr pp glucose rise above high target, 2 hr pp glucose rise between high and low target, and For 2 hr pp glucose rise below low target.

If the glucose rise at 2 hours was above high target for the previous day, then the new meal dose for the current day is calculated as a percent increase from the previous day's given dose by multiplying the previous day's given dose by a factor Y (that is >1.0), from the linear equation Y=mX+b, where X is the magnitude of glucose rise above the high target (glucose rise-target rise, always positive), b is the minimum increase factor for any glucose rise above the high target (1.05 for a 5% increase), and m is the slope of the line. In the exemplary embodiment, there is a set max percent increase, so for example, if this is set at 15%, then even if the calculation gives a result of Y=1.2, the recommended dose would still be to increase as if Y=1.15. The slope m of the line is determined by the “Advanced Settings”: X=post meal glucose over target at max, Y=1+meal max increase %/100, and b=1+meal min increase %/100. So, in this embodiment, the recommended dose=yesterday's dose×[meal increase slope×(2 hr pp glucose rise−high target)+meal increase y-intercept].

If the 2 hr pp glucose rise was between high and low target for the previous day, then, then there will be no change to the recommended dose.

If the 2 hr pp glucose rise was below low target for the previous day, then the new dose is calculated as a percent decrease from yesterday's dose calculated by multiplying yesterday's dose by a factor Y (that is between 0 and 1.0), from the linear equation Y=mx+b, where X is the magnitude of glucose rise below the low target (glucose rise-low target, always negative), b is the minimum decrease factor for any glucose rise below the low target (0.9 for a 10% decrease), and m is the slope of the line. In the exemplary embodiment, there is NO set max decrease, as this would put patient at risk for recurrent severe hypoglycemia. The intercept and slope of the line is set by values used during a pilot study conducted by the present inventors, where a 20% reduction in dose was made when 2 hr pp glucose rise was 36 mg/dL below the low target, with a minimum meal decrease factor (Y-intercept b) of 0.9, giving m=0.002778. So, in this embodiment, the recommended dose=yesterday's dose×[meal decrease slope×(2 hr pp glucose rise-low target)+meal decrease y-intercept].

Furthermore, in the exemplary embodiment, the present methodology includes a hypoglycemia override. In particular, if patient has a low glucose (below the input value on the settings page) between meals or before bedtime, and a dose reduction in the prior meal's dose was not already determined by 2 hr glucose or next premeal or bedtime glucose, then reduction in the prior meal's base dose will be calculated using the hypoglycemia override formula as follows: Y=0.01333X+0.9, with a 10% decrease for a glucose just below hypoglycemia value, and slope derived from a 30% decrease for a glucose 15 mg/dL below the hypoglycemia value.

Furthermore, according to a further aspect of this methodology, the following describes how the calculations would be performed for settings of dose increase frequency of 2 days or greater. Specifically, dose decreases are unchanged.

Dose increases are calculated by the average of the Y values over that period of days, with a glucose rise at target generating a Y value of 1.0. As values below target will result in a dose decrease that day, the day-clock starts over at that time. For example, if set to increase every 4 days, and since the last dose change, patient's 2 hr pp glucose rise was at goal once, and above high target 3 days, with Y values of 1.11, 1.15, and 1.13, then his base meal dose would be calculated based on formula from his settings page, with a Y of (1.0+1.1+1.15+1.13)/4=1.095.

### Other Scenarios

Moreover, of note, meals should be as consistent as possible during dose titration, with a similar number of servings of carbohydrates, and a similar time of day within a few hours. Meals should be at least 4 hours apart from each other. Carb snacks and drinks between meals and before bed should be avoided during dose titration. Three meals a day are recommended, but understand that some people usually eat four, or if they stopped snacking on carbs between meals, they would need a fourth meal in the late evening to match their current intake. For those selecting four meals/day, they will be labeled Breakfast, Lunch, Dinner, and Fourth Meal. New calorie restrictions or new attempts to lose weight during dose titration are not recommended.

### Missed Meal:

A patient may have fewer than their usual number of meals in a particular day. They may input their glucose level and arrow for a correction dose with arrow adjustment, and check NO for meal eaten using the titration application 50. A correction dose may be recommended, and they can document whether they gave this or another amount. No adjustment will be made to that meal's base dose for the following day. If they put in no info for this meal, and then log info for bedtime or for the next meal, then the app will assume no food was eaten and no insulin given. No adjustment will be made to that missed meal's base dose for the next day.

### Missed Basal:

A patient may forget to take, or forget to document taking, a dose of basal insulin. When this happens, the dose will not be adjusted the next day for a fasting glucose above target. Also meal doses the next day will not be adjusted for high post-prandial rises that occurred when basal may not have been on board. The total daily dose for correction calculations and arrow adjustment calculations will still include the recommended basal dose from that day.

### Not Checking 2 hr Postprandial Glucoses:

In a patient who unclicks “Checking 2 hour post-meal glucose” in the settings page, meal doses will be adjusted by the next meal's premeal glucose value, or the bedtime glucose value. The same formula will be used as for a 2 hr pp value, but with glucose above premeal high target or below premeal low target used instead of glucose rise above high target rise or below low target rise. So recommended dose=yesterday's dose×[meal increase slope×(next meal glucose−next meal high target)+meal increase y-intercept], or recommended dose=yesterday's dose×[meal decrease slope×(next meal glucose-low target)+meal decrease y-intercept].

### Missed 2 hr Glucose:

In a patient who is checking 2 hr pp glucoses, if they miss one, that meal's base dose will be adjusted the next day based on their next meal's premeal glucose, or bedtime glucose, as above.

### Alternative Implementation

In the embodiments described so far herein, basal dose is adjusted by a percentage based on how far above or below a target range the fasting glucose is according to y=mx+b formulas. According to an alternative embodiment, the user is given the option in the “Settings” page to choose a different basal target method wherein rather than being based on fasting glucose target range, basal dose is adjusted based on the fasting glucose change from bedtime glucose. Choosing this option would remove the fasting glucose “high target” and “low target” settings in general settings, and instead add “percent change in basal at 75 mg/dL fall overnight” in advanced settings. Choosing this alternative embodiment would be appropriate for providers treating patients with type 1 diabetes, pancreatectomy diabetes, or who otherwise make little to no insulin of their own, and are at risk of fasting hypoglycemia if the current basal dose is causing the glucose to significantly trend downward overnight. The basal dose would then be adjusted by a percentage calculated by the change in glucose between bedtime (or 4+ hours after last bolus) and before breakfast. Based on the formula below:

$$New basal = \frac{old basal}{1 + \frac{glucose drop}{[ 900 / 24 hr ] * time of drop} \text{?}}$$

$$\text{?} \text{indicates text missing or illegible when filed}$$

and assuming average 8 hr time of drop overnight, new basal equals old basal divided by [1+(drop/300)]. This line is nearly linear between drop of 0 and 150 mg/dL, with a basal reduction of 33.33% at a glucose fall of 150 mg/dL. So percentage change in basal, up or down, can be calculated using a line with slope of 0.002222. A provider may change this slope in “Advanced Settings”, by choosing a different % change in basal at 75 mg/dL overnight drop. Default will be 17% to match the above calculation and slope.

Of note is the fact that the formula above was derived based on the premise that when glucose is falling during fasting with only basal insulin on board and no endogenous insulin production, the correct (lower) basal insulin dose would have kept glucose steady, and the extra “basal” insulin that was received is acting as correction insulin. The units of extra insulin bringing the glucose down over the hours between glucose assessments is being derived from the assumed relationship between sensitivity to correction insulin and a patient's basal insulin requirements, using ISF=900/total daily basal. Including the number of hours over which the glucose fell, this is converted to a change in the 24 hr insulin dose. As this formula is not linear, it has been converted to nearest linear formula within range of overnight glucose excursions encountered, and allowed for provider to change the slope setting.

Extending the formula above into the range of glucose rising overnight would yield a steeper slope for dose increases, with a 33.33% increase in basal for a rise of 75 mg/dL overnight. As this overnight change method is being chosen to prevent fasting hypoglycemia in those susceptible to it, it would not be desirable to have more aggressive dose increases for overnight rise in glucose. This is another reason to use the linear formula and the same slope as for dose decreases when calculating dose increases for rising glucose overnight. On the correct stable basal insulin dose, glucose will be the same in the AM as it is 4 hours after last meal/correction bolus the day before.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A system for automatically prescribing and/or adjusting insulin doses for a patient, wherein a first titration application component is implemented on a patient computing device of the patient, wherein the first titration application component is structured and configured to log glucose level data measured from the patient and to log insulin dose data indicative of a number of insulin doses given to the patient, the system comprising:
a provider computing device of a healthcare provider of the patient, the provider computing device including a second titration application component structured to be implemented on the provider computing device, wherein the second titration application component is structured and configured to receive the glucose level data and the insulin dose data from the patient computing device, determine a correction factor based on at least a sensitivity constant specified by the healthcare provider, and determine the insulin dose recommendation for the patient based on the insulin dose data, the glucose level data and the correction factor, wherein the provider computing device is structured and configured to transmit the insulin dose recommendation to the patient computing device.

2. The system according to claim 1, wherein a linear equation is used to determine the insulin dose recommendation such that a previous day's dose is adjusted based on a slope and an intercept of the linear equation and maximum and minimum increase and decrease percentages specified by the healthcare provider using the second titration application component.

3. The system according to claim 1, wherein the first titration application component is structured and configured to log CGM trend arrow data indicative of a current trend in the patient's glucose levels, and wherein the insulin dose recommendation for the patient is based on the glucose level data, the insulin dose data, the correction factor, and the CGM trend arrow data.

4. The system according to claim 3, wherein the healthcare provider is able to specify that the patient practices carb counting using a carb ratio, and wherein the insulin dose recommendation for the patient is also based on a ratio of a meal carbohydrate estimation provided by the patient to the carb ratio.

5. The system according to claim 1, wherein the healthcare provider is able to specify a dose increase frequency using the second titration application component, and wherein if the dose increase frequency is 2 days or greater, the correction facto comprises an average of a plurality of calculated daily correction factors.

6. The system according to claim 1, wherein the insulin dose recommendation is a basal insulin dose, wherein the healthcare provider is able to specify the following using the second titration application component: a high target, a low target, a max basal increase %, and a min basal increase %, and wherein:
(i) if a fasting glucose of the patient as indicated by the glucose level data is above the high target, the insulin dose recommendation is determined based on the following:
previous day's dose as indicated by the insulin dose data×[basal increase slope×(the fasting glucose−the high target)+basal increase y-intercept], wherein the basal increase slope=1+(the max basal increase %)/100, and wherein the basal increase y-intercept=1+(min basal increase %)/100;
(ii) if the fasting glucose of the patient as indicated by the glucose level data is between the high target and the low target, the insulin dose recommendation equals the previous day's dose as indicated by the insulin dose data; and
(iii) if the fasting glucose of the patient as indicated by the glucose level data is below the low target, the insulin dose recommendation is determined based on the following:
previous day's dose as indicated by the insulin dose data×[basal decrease slope×(the fasting glucose−the low target)+basal decrease y-intercept], wherein the basal decrease slope and the basal decrease y-intercept are each predetermined values.

7. The system according to claim 6, wherein the insulin dose recommendation is determined based on a hypoglycemia override if the patient has a predetermined low glucose level between bedtime and fasting glucose level entry into the patient computing device.

8. The system according to claim 1, wherein the insulin dose recommendation includes a mealtime correction dose, wherein the healthcare provider is able to specify the following using the second titration application component: a correction target and the sensitivity constant, wherein the correction factor is a sensitivity factor determined from the sensitivity constant and wherein the mealtime correction dose=(a premeal glucose level as indicated by the insulin dose data−the correction target)/the sensitivity factor.

9. The system according to claim 8, wherein the sensitivity factor is determined as follows: the sensitivity constant/a total daily dose (TDD) for the patient as indicated by the insulin dose data.

10. The system according to claim 8, wherein the sensitivity factor is determined as follows: the sensitivity constant/a long-acting insulin dose.

11. The system according to claim 8, wherein the sensitivity factor is determined as follows: the sensitivity constant×a carb ratio used by the patient for carb counting.

12. The system according to claim 1, wherein the first titration application component is structured and configured to log CGM trend arrow data indicative of a current trend in the patient's glucose levels and estimates how high glucose will be in a predetermined number of minutes if nothing is done, wherein the healthcare provider is able to specify a sensitivity constant using the second titration application component, wherein the insulin dose recommendation includes an arrow adjustment dose, wherein the correction factor is a sensitivity factor determined from the sensitivity constant and wherein the arrow adjustment dose=a number of arrow increments indicated by the CGM trend arrow data×the predetermined number of minutes×the sensitivity factor.

13. The system according to claim 11, wherein the sensitivity factor is determined as follows: the sensitivity constant/a total daily dose (TDD) for the patient as indicated by the insulin dose data.

14. The system according to claim 11, wherein the sensitivity factor is determined as follows: the sensitivity constant/a long-acting insulin dose.

15. The system according to claim 11, wherein the sensitivity factor is determined as follows: the sensitivity constant×a carb ratio used by the patient for carb counting.

16. The system according to claim 8, wherein the insulin dose recommendation includes a sum of the mealtime correction dose and the arrow adjustment dose.

17. The system according to claim 12, wherein the insulin dose recommendation includes a sum of the mealtime correction dose and the arrow adjustment dose.

18. The system according to claim 1, wherein the insulin dose recommendation is a base dose for a meal on a given day, wherein the healthcare provider is able to specify the following using the second titration application component: a high target, a low target, a max meal increase %, and a min meal increase %, and wherein:
(i) if a 2 hr pp glucose rise of the patient as indicated by the glucose level data is above the high target, the insulin dose recommendation is determined based on the following:
a base dose for the meal on a day previous to the given day as indicated by the insulin dose data×[meal increase slope×(2 hr pp glucose rise−high target)+meal increase y-intercept], wherein the meal increase slope=1+(the max meal increase %)/100, and wherein the basal increase y-intercept=1+(min meal increase %)/100;
(ii) if the 2 hr pp glucose rise of the patient as indicated by the base dose is between the high target and the low target, the insulin dose recommendation equals the base dose; and
(iii) if the 2 hr pp glucose rise of the patient as indicated by the glucose level data is below the low target, the insulin dose recommendation is determined based on the following:
the base dose×[meal decrease slope×(the fasting glucose−the low target)+meal decrease y-intercept], wherein the meal decrease slope and the meal decrease y-intercept are each predetermined values.

19. A system for automatically prescribing and/or adjusting a basal insulin dose for a patient, wherein a first titration application component is implemented on a patient computing device of the patient, wherein the first titration application component is structured and configured to log: (i) existing basal dose data indicative of an existing basal dose for the patient, (ii) glucose change data for the patient indicative of a change between a bedtime glucose level of the patient and a breakfast glucose level of the patient, and (iii) time of drop data indicative of a time period over which the change occurred, the system comprising:
a provider computing device of a healthcare provider of the patient, the provider computing device including a second titration application component structured to be implemented on the provider computing device, wherein the second titration application component is structured and configured to receive the existing basal dose data, the glucose change data, and the time of drop data, determine an insulin dose recommendation for the patient based on existing basal dose data, the glucose change data and the time of drop data, and transmit the insulin dose recommendation to the patient computing device.

20. The system according to claim 19, wherein the insulin dose recommendation is a new basal dose calculated as follows: new basal dose=(existing basal dose data)/(1+(glucose change data/(a constant/24×time of drop data))).

21. The system according to claim 20, wherein the constant is 900.
